(19) United States
(12) Patent Application Publication
(10) Pub. No.: US 2022/0365049 A1
(43) Pub. Date: Nov. 17, 2022

(54) AUDIT LEDGER FOR EXTERNALITY TRACKING AND REPORTING

(71) Applicant: Microsoft Technology Licensing, LLC, Redmond, WA (US)

(72) Inventors: Mehmet Kadri UMAY, Redmond, WA (US); Jyothsna Devi BIJJAM, Redmond, WA (US); Imran SIDDIQUE, Bellevue, WA (US); Nayana Singh PATEL, Mercer Island, WA (US)

(21) Appl. No.: 17/317,320

(22) Filed: May 11, 2021

(51) Int. Cl. G01N 33/00

(52) U.S. Cl. CPC G01N 33/007; G01N 33/0075

(57) ABSTRACT

The disclosed technology is generally directed to an audit ledger for externality tracking. In one example of the technology, auditing of a virtual sensor is enabled. The virtual sensor is configured to output an externality value based on received telemetry data. The externality values are associated with the at least one particular type of quantifiable technical externality. Signing of the virtual sensor is enabled. Audit information associated with the signing of the virtual sensor is stored on a ledger. Periodic aggregated externality values based on the externality values output by the virtual sensor are calculated. The periodic aggregated externality values are stored on a distributed ledger. An audit of the periodic aggregated externality values is enabled. The auditing of the periodic aggregated externality values includes verifying the signing of the virtual sensor by the auditor based on the stored audit information.

### BACKGROUND

The energy industry is increasingly implementing measures to track greenhouse gas (GHG) emissions in an effort to reduce GHG emissions worldwide. Such emissions may include, for example, carbon dioxide (CO₂), methane (CH₄) and other hydrofluorocarbons (HFCs), perfluorocarbons (PFCs), sulfur hexafluoride (SF₆), and nitrogen trifluoride (NF₃), as examples. Likewise, other industries, such as agriculture, forestry, and GHG capture initiatives, are growing as offsetting technologies. Currently, GHG emissions are typically reported to various regulators annually, and report generation is commonly done though manual processes that may be inefficient and prone to error.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Briefly stated, the disclosed technology is generally directed to an audit ledger for externality tracking. In some examples, telemetry data that is associated with at least one particular type of quantifiable technical externality is received. In some examples, auditing of a first virtual sensor by an auditor is enabled. In some examples, the first virtual sensor is configured to output at least a first externality value of a plurality of externality values based on the received telemetry data. In some examples, each externality value of the plurality of externality values is a value that is associated with the at least one particular type of quantifiable technical externality. In some examples, signing of the first virtual sensor by the auditor with an auditor key that is associated with the auditor responsive to the auditing of the first virtual sensor being successful is enabled. In some examples, virtual sensor audit information that is associated with the signing of the first virtual sensor is stored on a first ledger. In some examples, periodic aggregated externality values are calculated based on the externality values output by the first virtual sensor. In some examples, the periodic aggregated externality values are stored on a second ledger. In some examples, the second ledger is a distributed ledger. In some examples, an audit of the periodic aggregated externality values is enabled. In some examples, the auditing of the periodic aggregated externality values includes verifying the signing of the first virtual sensor by the auditor based, at least in part, on the stored virtual sensor audit information.

Other aspects of and applications for the disclosed technology will be appreciated upon reading and understanding the attached figures and description.

### BRIEF DESCRIPTION OF THE DRAWINGS

Non-limiting and non-exhaustive examples of the present disclosure are described with reference to the following drawings. In the drawings, like reference numerals refer to like parts throughout the various figures unless otherwise specified. These drawings are not necessarily drawn to scale.

For a better understanding of the present disclosure, reference will be made to the following Detailed Description, which is to be read in association with the accompanying drawings, in which:

FIG. 1 is a block diagram illustrating one example of a suitable environment in which aspects of the technology may be employed;

FIG. 2 is a block diagram illustrating one example of a suitable computing device according to aspects of the disclosed technology;

FIG. 3 is a block diagram illustrating an example of a network-connected system;

FIG. 4 is a block diagram illustrating an example of a system for externality tracking;

FIG. 5 is a block diagram illustrating an example of a system for GHG emission reporting and tracking; and

FIG. 6 is a flow diagram illustrating an example process for externality tracking, in accordance with aspects of the present disclosure.

### DETAILED DESCRIPTION

The following description provides specific details for a thorough understanding of, and enabling description for, various examples of the technology. One skilled in the art will understand that the technology may be practiced without many of these details. In some instances, well-known structures and functions have not been shown or described in detail to avoid unnecessarily obscuring the description of examples of the technology. It is intended that the terminology used in this disclosure be interpreted in its broadest reasonable manner, even though it is being used in conjunction with a detailed description of certain examples of the technology. Although certain terms may be emphasized below, any terminology intended to be interpreted in any restricted manner will be overtly and specifically defined as such in this Detailed Description section. Throughout the specification and claims, the following terms take at least the meanings explicitly associated herein, unless the context dictates otherwise. The meanings identified below do not necessarily limit the terms, but merely provide illustrative examples for the terms. For example, each of the terms “based on” and “based upon” is not exclusive, and is equivalent to the term “based, at least in part, on,” and includes the option of being based on additional factors, some of which may not be described herein. As another example, the term “via” is not exclusive, and is equivalent to the term “via, at least in part,” and includes the option of being via additional factors, some of which may not be described herein. The meaning of “in” includes “in” and “on.” The phrase “in one embodiment,” or “in one example,” as used herein does not necessarily refer to the same embodiment or example, although it may. Use of particular textual numeric designators does not imply the existence of lesser-valued numerical designators. For example, reciting “a widget selected from the group consisting of a third foo and a fourth bar” would not itself imply that there are at least three foo, nor that there are at least four bar, elements. References in the singular are made merely for clarity of reading and include plural references unless plural references are specifically excluded. The term “or” is an inclusive “or” operator unless specifically indicated otherwise. For example, the phrases “A or B” means “A, B, or A and B.” As used herein, the terms “component” and “system” are intended to encompass hardware, software, or various combinations of hardware and software. Thus, for example, a system or component may be a process, a process executing on a computing device, the computing device, or a portion thereof. The term “cloud” or “cloud computing” refers to shared pools of configurable computer system resources and higher-level services over a wide-area network, typically the Internet. “Edge” devices refer to devices that are not themselves part of the cloud, but are devices that serve as an entry point into enterprise or service provider core networks.

Briefly stated, the disclosed technology is generally directed to an audit ledger for externality tracking. In some examples, telemetry data that is associated with at least one particular type of quantifiable technical externality is received. In some examples, auditing of a first virtual sensor by an auditor is enabled. In some examples, the first virtual sensor is configured to output at least a first externality value of a plurality of externality values based on the received telemetry data. In some examples, each externality value of the plurality of externality values is a value that is associated with the at least one particular type of quantifiable technical externality. In some examples, signing of the first virtual sensor by the auditor with an auditor key that is associated with the auditor responsive to the auditing of the first virtual sensor being successful is enabled. In some examples, virtual sensor audit information that is associated with the signing of the first virtual sensor is stored on a first ledger. In some examples, periodic aggregated externality values are calculated based on the externality values output by the first virtual sensor. In some examples, the periodic aggregated externality values are stored on a second ledger. In some examples, the second ledger is a distributed ledger. In some examples, an audit of the periodic aggregated externality values is enabled. In some examples, the auditing of the periodic aggregated externality values includes verifying the signing of the first virtual sensor by the auditor based, at least in part, on the stored virtual sensor audit information.

A distributed audit ledger may be used for the tracking and reporting of quantifiable technical externalities, such as pollution. For instance, in some examples, the distributed audit ledger is used to track GHG emissions. GHG emissions tracking may be tracked as carbon equivalents and referred to as carbon emissions tracking. In some examples, the audit ledger is a cross-company ledger that may be used for the tracking and reporting of emissions across supply chains.

One or more virtual sensor boxes may be used to determine GHG emissions (or other tracked externality values). In some examples, the flow of GHG emissions is not tracked directly. Instead, in these examples, the GHG emissions are determined based on a model and parameters in a virtual sensor box that receives input telemetry data, the model and parameters are used to calculate the GHG emission based on the received telemetry data. The model, parameters, and type of telemetry data received may vary by context. For example, different models, parameters, and types of telemetry data may be used to determine the GHG emissions from cement manufacturing than are used to determine the GHG emissions from power generation.

In some examples, the auditor may audit a virtual sensor box and, and then seal the virtual sensor box by signing the virtual sensor box with an auditor key if the auditing is successful. The sealing event along with a timestamp may be sent to a central distributed ledger. In some examples, the sealing event can then be audited at a later time.

Periodic aggregated GHG emissions values, such as daily aggregated GHG emissions values, may be archived and stored locally in a ledger. The daily aggregated emission value, the model and the parameters, the location and hash of the archive file with time series data may be stored in the central distributed ledger. In some examples, this manner of storing information, signing, and auditor provides credibility to reported emissions information. Each emission value involved in a transaction may also be stored with a unique transaction identifier (ID) associated with the transaction. In some examples, the central ledger may be used across one or more supply chains, and the emissions information may be used to track and report scope three emissions data. In some examples, scope three emissions data includes emissions that result from activities from assets not owned or controlled by the reporting organization, but that the organization indirectly impacts in its supply chain.

### Illustrative Devices/Operating Environments

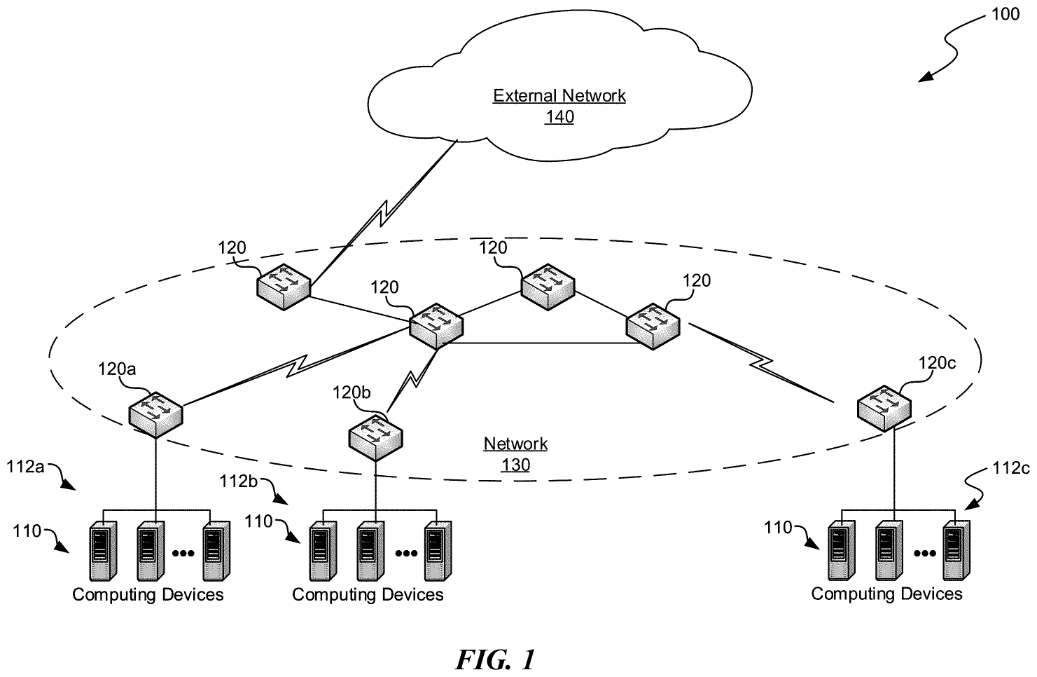

FIG. 1 is a diagram of environment 100 in which aspects of the technology may be practiced. As shown, environment 100 includes computing devices 110, as well as network nodes 120, connected via network 130. Even though particular components of environment 100 are shown in FIG. 1, in other examples, environment 100 can also include additional and/or different components. For example, in certain examples, the environment 100 can also include network storage devices, maintenance managers, and/or other suitable components (not shown). Computing devices 110 shown in FIG. 1 may be in various locations, including on premise, in the cloud, or the like. For example, computer devices 110 may be on the client side, on the server side, or the like.

As shown in FIG. 1, network 130 can include one or more network nodes 120 that interconnect multiple computing devices 110, and connect computing devices 110 to external network 140, e.g., the Internet or an intranet. For example, network nodes 120 may include switches, routers, hubs, network controllers, or other network elements. In certain examples, computing devices 110 can be organized into racks, action zones, groups, sets, or other suitable divisions. For example, in the illustrated example, computing devices 110 are grouped into three host sets identified individually as first, second, and third host sets 112a-112c. In the illustrated example, each of host sets 112a-112c is operatively coupled to a corresponding network node 120a-120c, respectively, which are commonly referred to as “top-of-rack” or “TOR” network nodes. TOR network nodes 120a-120c can then be operatively coupled to additional network nodes 120 to form a computer network in a hierarchical, flat, mesh, or other suitable types of topology that allows communications between computing devices 110 and external network 140. In other examples, multiple host sets 112a-112c may share a single network node 120. Computing devices 110 may be virtually any type of general- or specific-purpose computing device. For example, these computing devices may be user devices such as desktop computers, laptop computers, tablet computers, display devices, cameras, printers, or smartphones. However, in a data center environment, these computing devices may be server devices such as application server computers, virtual computing host computers, or file server computers. Moreover, computing devices 110 may be individually configured to provide computing, storage, and/or other suitable computing services.

In some examples, one or more of the computing devices 110 is a device that is configured to be part of a system for externality tracking.

### Illustrative Computing Device

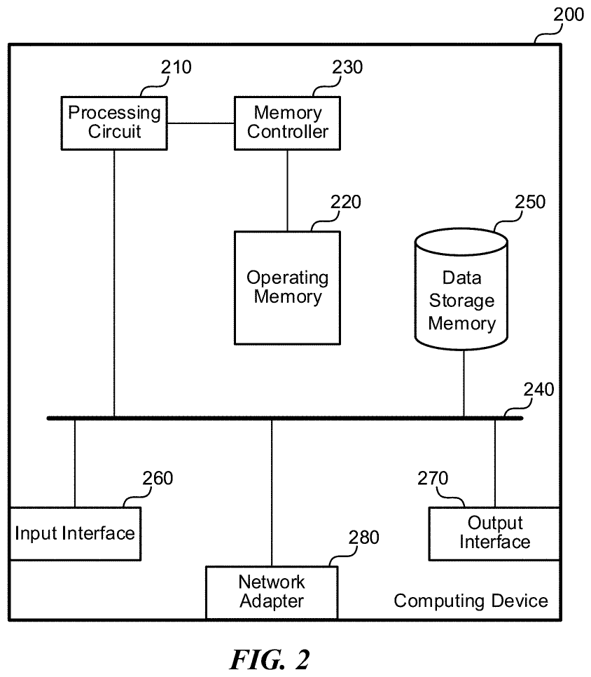

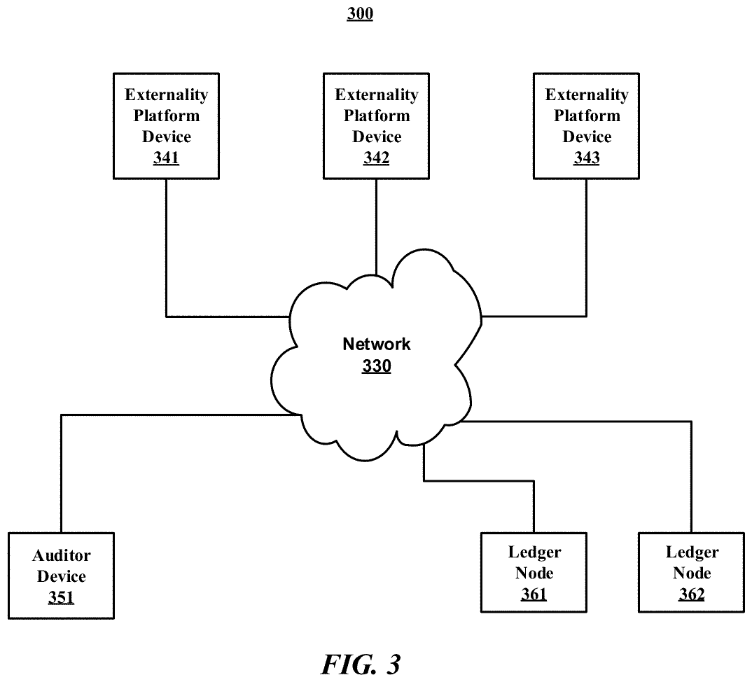

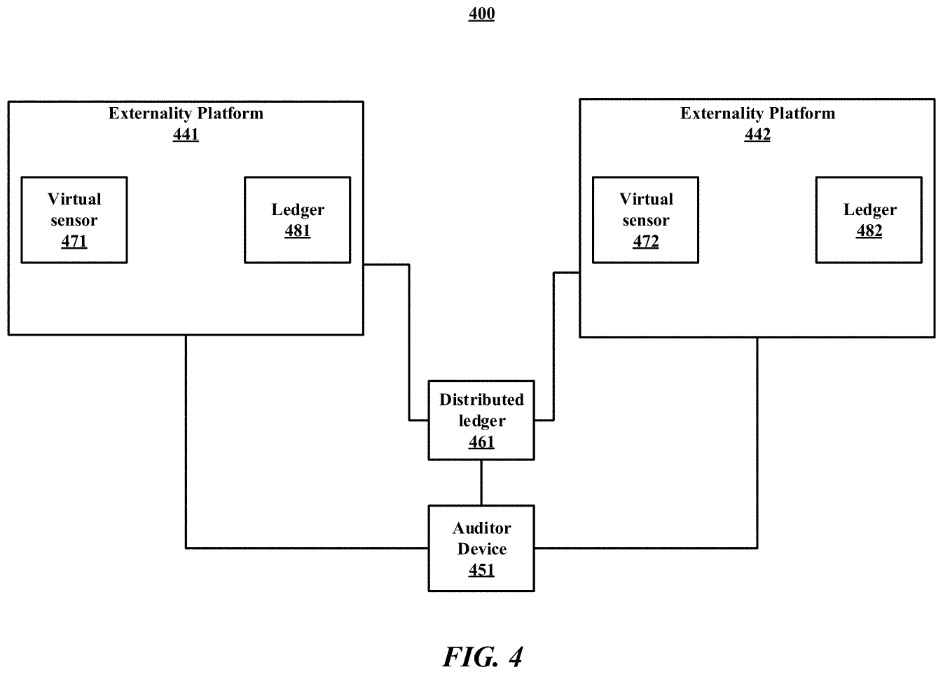

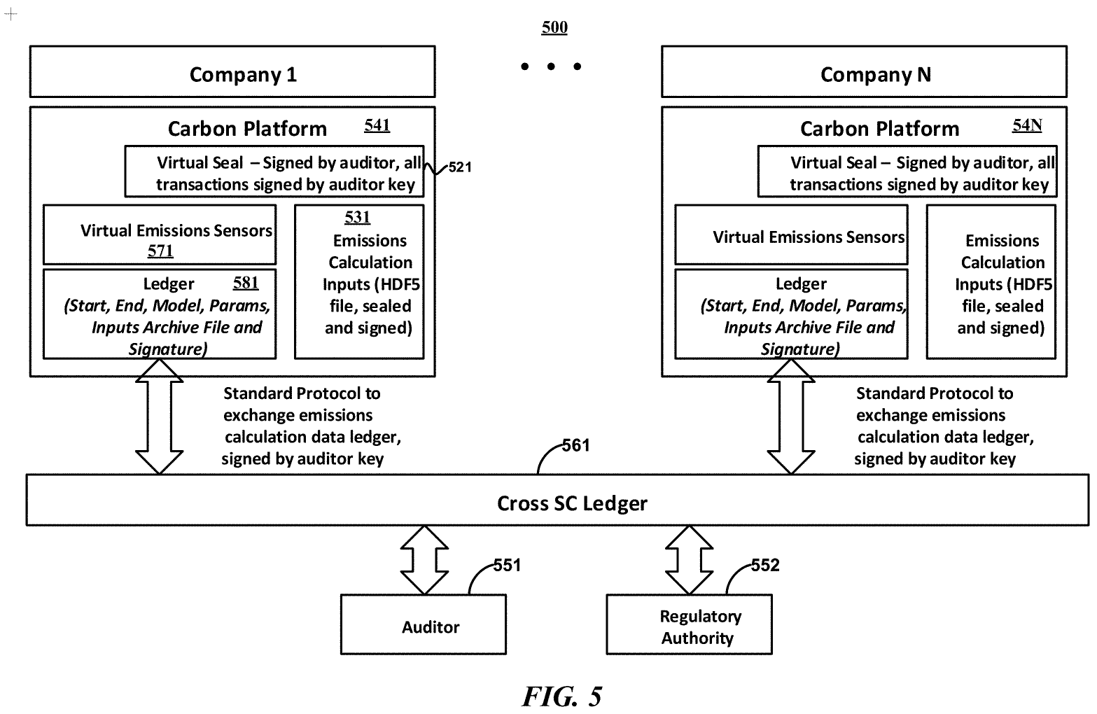

FIG. 2 is a diagram illustrating one example of computing device 200 in which aspects of the technology may be practiced. Computing device 200 may be virtually any type of general- or specific-purpose computing device. For example, computing device 200 may be a user device such as a desktop computer, a laptop computer, a tablet computer, a display device, a camera, a printer, or a smartphone. Likewise, computing device 200 may also be a server device such as an application server computer, a virtual computing host computer, or a file server computer, e.g., computing device 200 may be an example of computing device 110 or network node 120 of FIG. 1. Likewise, computer device 200 may be an example any of the devices, a device within any of the distributed systems, illustrated in or referred to in FIG. 3, FIG. 4, and/or FIG. 5, as discussed in greater detail below. As illustrated in FIG. 2, computing device 200 includes processing circuit 210, operating memory 220, memory controller 230, data storage memory 250, input interface 260, output interface 270, and network adapter 280. Each of these afore-listed components of computing device 200 includes at least one hardware element.

Computing device 200 includes at least one processing circuit 210 configured to execute instructions, such as instructions for implementing the herein-described workloads, processes, or technology. Processing circuit 210 may include a microprocessor, a microcontroller, a graphics processor, a coprocessor, a field-programmable gate array, a programmable logic device, a signal processor, or any other circuit suitable for processing data. The aforementioned instructions, along with other data (e.g., datasets, metadata, operating system instructions, etc.), may be stored in operating memory 220 during run-time of computing device 200. Operating memory 220 may also include any of a variety of data storage devices/components, such as volatile memories, semi-volatile memories, random access memories, static memories, caches, buffers, or other media used to store run-time information. In one example, operating memory 220 does not retain information when computing device 200 is powered off. Rather, computing device 200 may be configured to transfer instructions from a non-volatile data storage component (e.g., data storage component 250) to operating memory 220 as part of a booting or other loading process. In some examples, other forms of execution may be employed, such as execution directly from data storage component 250, e.g., eXecute In Place (XIP).

Operating memory 220 may include 4^{th} generation double data rate (DDR4) memory, 3^{rd} generation double data rate (DDR3) memory, other dynamic random access memory (DRAM), High Bandwidth Memory (HBM), Hybrid Memory Cube memory, 3D-stacked memory, static random access memory (SRAM), magnetoresistive random access memory (MRAM), pseudorandom random access memory (PSRAM), or other memory, and such memory may comprise one or more memory circuits integrated onto a DIMM, SIMM, SODIMM, Known Good Die (KGD), or other packaging. Such operating memory modules or devices may be organized according to channels, ranks, and banks. For example, operating memory devices may be coupled to processing circuit 210 via memory controller 230 in channels. One example of computing device 200 may include one or two DIMMs per channel, with one or two ranks per channel. Operating memory within a rank may operate with a shared clock, and shared address and command bus. Also, an operating memory device may be organized into several banks where a bank can be thought of as an array addressed by row and column. Based on such an organization of operating memory, physical addresses within the operating memory may be referred to by a tuple of channel, rank, bank, row, and column.

Despite the above-discussion, operating memory 220 specifically does not include or encompass communications media, any communications medium, or any signals per se.

Memory controller 230 is configured to interface processing circuit 210 to operating memory 220. For example, memory controller 230 may be configured to interface commands, addresses, and data between operating memory 220 and processing circuit 210. Memory controller 230 may also be configured to abstract or otherwise manage certain aspects of memory management from or for processing circuit 210. Although memory controller 230 is illustrated as single memory controller separate from processing circuit 210, in other examples, multiple memory controllers may be employed, memory controller(s) may be integrated with operating memory 220, or the like. Further, memory controller(s) may be integrated into processing circuit 210. These and other variations are possible.

In computing device 200, data storage memory 250, input interface 260, output interface 270, and network adapter 280 are interfaced to processing circuit 210 by bus 240. Although, FIG. 2 illustrates bus 240 as a single passive bus, other configurations, such as a collection of buses, a collection of point-to-point links, an input/output controller, a bridge, other interface circuitry, or any collection thereof may also be suitably employed for interfacing data storage memory 250, input interface 260, output interface 270, or network adapter 280 to processing circuit 210.

In computing device 200, data storage memory 250 is employed for long-term non-volatile data storage. Data storage memory 250 may include any of a variety of non-volatile data storage devices/components, such as non-volatile memories, disks, disk drives, hard drives, solid-state drives, or any other media that can be used for the non-volatile storage of information. However, data storage memory 250 specifically does not include or encompass communications media, any communications medium, or any signals per se. In contrast to operating memory 220, data storage memory 250 is employed by computing device 200 for non-volatile long-term data storage, instead of for run-time data storage.

Also, computing device 200 may include or be coupled to any type of processor-readable media such as processor-readable storage media (e.g., operating memory 220 and data storage memory 250) and communication media (e.g., communication signals and radio waves). While the term processor-readable storage media includes operating memory 220 and data storage memory 250, the term “processor-readable storage media,” throughout the specification and the claims whether used in the singular or the plural, is defined herein so that the term “processor-readable storage media” specifically excludes and does not encompass communications media, any communications medium, or any signals per se. However, the term “processor-readable storage media” does encompass processor cache, Random Access Memory (RAM), register memory, and/or the like.

Computing device 200 also includes input interface 260, which may be configured to enable computing device 200 to receive input from users or from other devices. In addition, computing device 200 includes output interface 270, which may be configured to provide output from computing device 200. In one example, output interface 270 includes a frame buffer, graphics processor, graphics processor or accelerator, and is configured to render displays for presentation on a separate visual display device (such as a monitor, projector, virtual computing client computer, etc.). In another example, output interface 270 includes a visual display device and is configured to render and present displays for viewing. In yet another example, input interface 260 and/or output interface 270 may include a universal asynchronous receiver/transmitter (UART), a Serial Peripheral Interface (SPI), Inter-Integrated Circuit (I2C), a General-purpose input/output (GPIO), and/or the like. Moreover, input interface 260 and/or output interface 270 may include or be interfaced to any number or type of peripherals.

In the illustrated example, computing device 200 is configured to communicate with other computing devices or entities via network adapter 280. Network adapter 280 may include a wired network adapter, e.g., an Ethernet adapter, a Token Ring adapter, or a Digital Subscriber Line (DSL) adapter. Network adapter 280 may also include a wireless network adapter, for example, a Wi-Fi adapter, a Bluetooth adapter, a ZigBee adapter, a Long-Term Evolution (LTE) adapter, SigFox, LoRa, Powerline, or a 5G adapter.

Although computing device 200 is illustrated with certain components configured in a particular arrangement, these components and this arrangement are merely one example of a computing device in which the technology may be employed. In other examples, data storage memory 250, input interface 260, output interface 270, or network adapter 280 may be directly coupled to processing circuit 210, or be coupled to processing circuit 210 via an input/output controller, a bridge, or other interface circuitry. Other variations of the technology are possible.

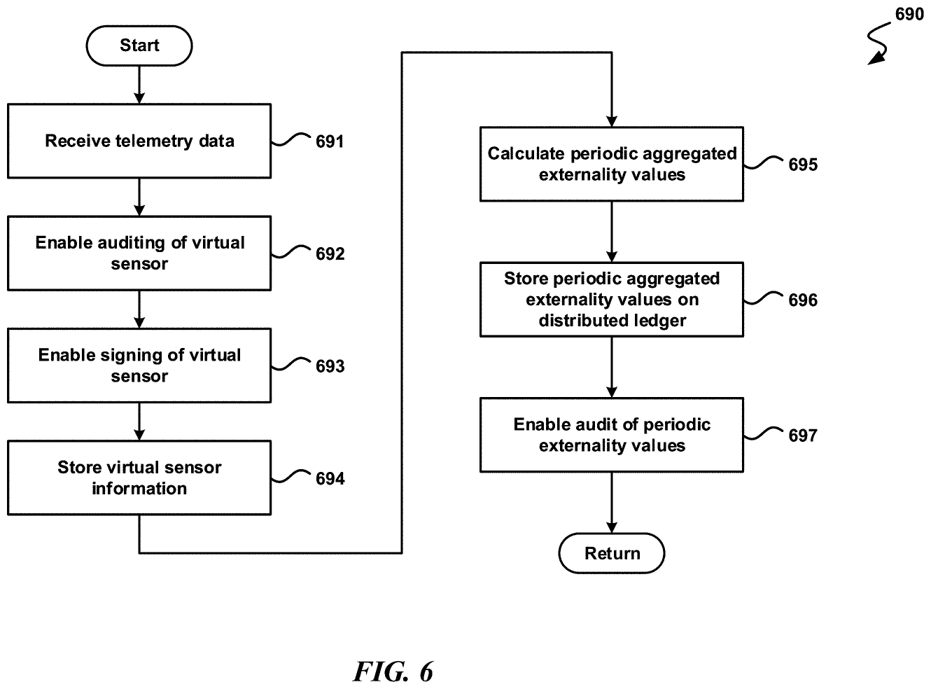

Some examples of computing device 200 include at least one memory (e.g., operating memory 220) adapted to store run-time data and at least one processor (e.g., processing unit 210) that is adapted to execute processor-executable code that, in response to execution, enables computing device 200 to perform actions, where the actions may include, in some examples, actions for one or more processes described herein, such as, in one example, process 690 of FIG. 6, which is discussed in greater detail below.

### Illustrative System

FIG. 3 is a block diagram illustrating an example of a system (300). System 300 may include network 330, as well as externality platform devices 341, 342, and 343; auditor device 351; and ledger nodes 361 and 362, which, in some examples, all connect to network 330.

Each of externality platform devices 341, 342, and 343; auditor device 351; and ledger nodes 361 and 362 may include examples of computing device 200 of FIG. 2. FIG. 3 and the corresponding description of FIG. 3 in the specification illustrates an example system for illustrative purposes that does not limit the scope of the disclosure.

In some examples, externality platform devices 341, 342, and 343 are part or all of one or more distributed systems that are configured to act as externality platforms, such as carbon emissions platforms, for one or more companies or other organizations. Auditor device 351 may be a device used by an auditor for functions associated with auditing or one or more aspects of externality tracking, such as carbon emissions tracking. Ledger nodes 361 may be nodes used for a distributed ledger. In some examples, one or more distributed systems that includes externality platform devices 341, 342, and 343 performs actions, where the actions may include, in some examples, actions for one or more processes described herein, such as, in one example, process 690 of FIG. 6, which is discussed in greater detail below.

Network 330 may include one or more computer networks, including wired and/or wireless networks, where each network may be, for example, a wireless network, local area network (LAN), a wide-area network (WAN), and/or a global network such as the Internet. On an interconnected set of LANs, including those based on differing architectures and protocols, a router acts as a link between LANs, enabling messages to be sent from one to another. Also, communication links within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communications links known to those skilled in the art. Furthermore, remote computers and other related electronic devices could be remotely connected to either LANs or WANs via a modem and temporary telephone link. Network 330 may include various other networks such as one or more networks using local network protocols such as 6LoWPAN, ZigBee, or the like. In essence, network 330 includes any communication method by which information may travel among externality platform devices 341, 342, and 343; auditor device 351; and ledger nodes 361 and 362. Although each device is shown connected as connected to network 330, that does not mean that each device communicates with each other device shown. In some examples, some devices shown only communicate with some other devices/services shown via one or more intermediary devices. Also, although network 330 is illustrated as one network, in some examples, network 330 may instead include multiple networks that may or may not be connected with each other, with some of the devices shown communicating with each other through one network of the multiple networks and other of the devices shown instead communicating with each other with a different network of the multiple networks.

System 300 may include more or less devices than illustrated in FIG. 3, which is shown by way of example only.

FIG. 4 is a block diagram illustrating an example of a system (400) for externality tracking. System 400 may be an example of system 300 of FIG. 3, or vice versa. System 400 may include externality platforms 441 and 442, auditor device 451, and distributed ledger 461. In some examples, each of the externality platforms (e.g., 441 and 442) includes or is part of one or more distributed systems. Externality platforms 441 and 442 may include examples of externality platform devices 351 and 352 of FIG. 3.

System 400 may be used for the tracking, reporting, and/or auditing of data associated with one or more tracked externalities. The tracked externalities may include at least one particular quantifiable technical externality, such as one or more specific types of pollutions or pollutants. In some examples, a portion of each of the externality platforms is on the edge, and a portion of each of the externality platforms is on the cloud. In some examples, each externality platform is associated with a particular entity, such as a company or organization, for tracking the tracked externalities for that company.

In some examples, distributed ledger 461 is used to track externality information for the tracked externality or tracked externalities across each of the entities, and distributed ledger 461 may also store information that be used for subsequent auditing of the externality information. In some examples, auditor device 451 may be used by an auditor to perform one or more auditing functions, such as auditing virtual sensors (e.g., one or more of virtual sensor 471 and 472), auditing of the determined externality values, and/or the like.

In some examples, each of the virtual sensors (e.g., virtual sensors 471 and 472) may be used to determine the externality values. For instance, in some examples, the externality values are carbon emissions. In some examples, the externality values are not tracked directly. Instead, in these examples, the externality values are determined based on a model and parameters in a virtual sensor (e.g., virtual sensor 471 or 472) that receives input telemetry data and uses the model and parameters to calculate the externality value based on the received telemetry data. The model, parameters, and type of telemetry data received may vary by context.

In some examples, an auditor may use auditor device 451 to audit a virtual sensor (e.g., virtual sensor 471 and/or 472) and then seal the virtual sensor by signing the virtual sensor with an auditor key if the auditing is successful. The sealing event with a timestamp may be sent to distributed ledger 461. In some examples, the sealing event can then be audited, by the auditor or by another auditing entity such as a regulatory authority, at a later time.

In some examples, the auditor may audit a virtual sensor by verifying the models and parameters used by the virtual sensor, and by testing the virtual sensor by providing one or more reference inputs and verifying that the outputs match those that should be provided based on the reference inputs provided.

Periodic aggregated externality values, such as daily aggregated externality values, may be archived and stored locally in a ledger (e.g., ledger 481 or ledger 482). The daily aggregated externality value, the model and the parameters, the location and hash of the archive file with time series data may be stored in distributed ledger 461. Each externality value involved in a transaction may also be stored with a unique transaction ID associated with the transaction. In some examples, distributed ledger 461 may be used across one or more supply chains, and the externality information may be used to track and report scope three externality data.

In various examples, the precise data stored in the local ledger (e.g., ledger 481 or ledger 482) versus distributed ledger 461 may vary in different examples. In some examples, the data is all stored in distributed ledger 461, and there is no separate local ledger (such as ledger 481). In some examples, various periodic aggregated externality values may all be stored in the local ledger, all be stored in distributed ledger 461, or a portion may be stored in the local ledger and another portion stored in distributed ledger 461. In some examples, not all of the data can be stored in distributed ledger 461, but data stored in the local ledger can be provided on an as-needed basis, such as if needed for an audit, or to determine scope three externality data.

In some examples, the scope one, scope two, and scope three externality data can be tracked for each of the entities for which externality values are being tracked. In some examples, scope one externality data includes externalities that occur from sources that are controlled or owned by an entity. In some examples, scope two externality data includes indirect externalities associated with the purchase of electricity, steam, heat, or cooling as a result of the entity's energy use. In some examples, scope three externality data includes externalities that result from activities from assets not owned or controlled by the reporting entity, but that the entity indirectly impacts in its supply chain. In some examples, entities can report their scope one, scope two, and/or scope three externality values, and those reported scope one, scope two, and/or scope three externality values can be audited by the auditor, a regulatory authority, or other suitable entity.

In some examples, the externality values associated with the tracked externalities can be positive or negative. For example, if carbon emissions are the tracked externality, then both carbon emissions, recorded as positive values, as well as carbon sequestrations, recorded as negatives values, can be tracked. Alternatively, emissions could be recorded as negative values with sequestrations recorded as positive values in some examples. In some examples, externality trading may also be tracked. For instance, in examples where the tracked externality is carbon emissions, entities that provide carbon sequestration may sell carbon credits to other entities, so that entities can buy carbon credits to reduce their carbon footprint. Examples of carbon sequestration may include direct air capture, carbon capture in underground storage, and other suitable methods that retrieve greenhouse gasses from the environment and store it safely and securely. In some examples, credits can be bought and sold for quantifiable technical externalities other than greenhouse gas emissions.

In some examples, the information stored in distributed ledger 461 is immutable. The immutability can be achieved in different ways in different examples. In some examples, the information is immutable not in the sense that it cannot be changed, but that if the information is changed, it is detectable that the information has been changed, effectively invalidating the information. In some examples, the immutability is accomplished by digital signing. In some examples, the digital signature can be used to verify that the information has not been changed. In some examples, the digital signature can also be used to verify the entity that performed the digital signing.

The tracking, reporting, and auditing of externality values enabled by system 400 may be used for various purposes. In some examples, entities may be subject to a cap for one or more negative externalities, or be required to maintained neutrality with respect to one or more negative externalities. Such a requirement might be required by regulation, mandated by an organization which the entity has voluntarily joined, or the like. System 400 may also be used for either voluntary or mandated disclosure of negative externality values in a credible manner using system 400.

In some examples, system 400 may also be used to facilitate the buying and selling of externality credits so that entities can either acquire neutrality with regard to a negative externality or not exceed a cap with regard to the negative externality, and to provide an incentive for entities that offset externalities to sell such externality credits. System 400 may also be used to select supplies with lower externalities, in order to reduce scope two and scope three emissions. In some examples, the tracking of externalities can also be used to detect anomalies, such as leaks. In some examples, the tracking of externalities can be used to assist in optimizing processing, such as reducing quantities associated with negative externalities and/or increasing quantities associated with positive externalities. In some examples, the externalities tracked are greenhouses gasses. In some examples, the externalities may include pollutants other than or in addition to greenhouse gasses. In some examples, suitable externalities other than pollutants may be tracked in addition to or instead of pollutants.

System 400 may include more or less devices than illustrated in FIG. 4, which is shown by way of example only.

FIG. 5 is a block diagram illustrating an example of a system (500) for GHG emissions reporting and tracking. System 400 may be an example of system 300 of FIG. 3 and/or system 400 of FIG. 4. System 500 may include carbon platforms 541-54N associated with a respective one of companies 1 through N, cross-supply-chain ledger 561, auditor device 551, and regulatory authority device 552. Carbon platform 541 may include virtual emission sensors 571, ledger 581, virtual seal 521, and emissions calculation inputs 531. Each of the other carbon platforms 552-55N may include similar components as carbon platform 551.

System 500 may be used for the tracking, reporting, and/or auditing of data associated with carbon emissions. In some examples, a portion of each of the carbon platforms 541N-54N is on the edge, and a portion of each of the carbon platforms 541-54N is on the cloud. In some examples, cross-supply-chain ledger 561 is used to track carbon emissions information across each of the companies Company 1 through Company N, and cross-supply-chain ledger 561 may also store information that can be used for subsequent auditing of the carbon emissions information. In some examples, auditor device 551 may be used by an auditor and/or regulatory authority device 552 may be used by a regulatory authority to perform one or more auditing functions, such as auditing the virtual sensors of the carbon platforms, auditing of the determined carbon emissions values, and/or the like. In some examples, cross-supply-chain ledger 561 is managed by the auditor, the regulatory authority, a standards body, and/or the like.

In some examples, carbon platform 541 stores emissions calculation inputs 531. In some examples, emissions calculations inputs are stored in a Hierarchical Data Format Version 5 (HDF5) format that is sealed and signed by Company 1. Suitable formats other than HDF5 may be used in other examples. In some examples, emissions calculations inputs 531 are generated based on telemetry data received by carbon platform 551 from physical sensors at one or more sites associated with Company 1. In various examples, the physical sensors may include chemical sensors (e.g., gas sensors), flow rate sensors, image sensors, light sensors, location sensors, motion sensors, pressure sensors, sound sensors, temperature sensors, and/or other suitable sensors. In some examples, in addition to telemetry data, emissions calculation inputs 531 may also include suitable data other than the telemetry for use in calculating the carbon emissions. In some examples, emissions calculations inputs 531 is data from which carbon emissions can be calculated.

In some examples, each of the virtual emissions sensors 571 on carbon platform 541 is configured to receive emissions calculation inputs 531 and to output carbon emissions data from the emissions calculation inputs 531. As discussed above, carbon emissions data may refer to GHG emissions data that is converted to a carbon equivalent. In some examples, each type of GHG has a corresponding carbon equivalent to which that type of GHG data is converted to carbon to determine the carbon equivalent. In some examples, the carbon emissions may be positive or negative, with negative emissions being carbon sequestration or the like.

In some examples, the carbon emissions values are determined based on a model and parameters that receive the emissions calculation inputs 531 and output the carbon emissions values. The model, parameters, and type of emissions calculation inputs may vary by context. For example, different models, parameters, and types of emissions calculation inputs may be used to determine the carbon emissions from cement manufacturing than are used to determine the carbon emissions from power generation. For instance, for cement manufacturing, the emissions calculation inputs 531 may include the data such as the heat of the kiln, the type of limestone, and other operating parameters that define the chemical reaction.

In some examples, each virtual emissions sensor may output the carbon emissions once every particular time interval. The time interval may vary in various examples, such as once per second in some examples, once per day in some other examples, or other suitable time interval in various other examples. In some examples, a virtual emissions sensor may calculate the carbon emissions at the rate of received telemetry, and in other examples, a virtual emissions sensor may calculate the carbon emissions at a rate that is slower than the rate of received telemetry.

In some examples, 581 is a local ledger for carbon platform 541 that is used to store some or all of the following: the inputs to the virtual emissions sensors 571, the outputs of the virtual emissions sensors 571, models and parameters of the virtual emission sensors 571, and/or the input archives files stored in 531, digitally signed by Company 1. In some examples, each time Company 1 makes a financial transaction that inputs GHG emissions at any scope, the corresponding emissions values are stored in ledger 581 with a transaction ID, where there is a unique transaction ID for each transaction. In some examples, a periodic emissions value, such as the daily aggregated emission value in some examples, is also determined and archived in ledger 581. In some examples, some of the information stated above as stored in ledger 581 may sent to and stored in cross-supply-chain ledger 561 instead of or in addition to ledger 581. In some examples, the virtual emissions sensors 571 calculate all of the scope one carbon emissions associated with Company 1.

In some examples, the information stored in ledger 581 is archived for a predetermined amount of time, such as a predetermined number of years. In some examples, the minimum time at which the information in ledger 581 must be archived in ledger 581 may be mandated by regulation or the like.

In some examples, ledgers such as the local ledgers (e.g., 581) and/or cross-supply-chain ledger 561 can be accessed by authorized entities through software developments kits (SDKs) and/or through application programming interfaces (APIs). For instance, in some examples, a standard API can be defined and published, so that any authorized entity that wishes to build an application to read data from a ledger can independently build those application using the published APIs. In some examples, authorized entities can be provided with an endpoint for the ledger and with anything else such as credentials, tokens, or the like needed to connect to the endpoint. In some examples, this can be done programmatically by providing an SDK layer on top of the API.

In some examples, the auditor may use auditor device 551 to audit each of the virtual emissions sensors. For instance, in some examples, the auditor may audit a virtual emissions sensor by verifying the models and parameters used by the virtual emissions sensor, and by testing the virtual emissions sensor by providing one or more reference inputs to the virtual emissions sensor and verifying that the outputs match those that should be provided based on the reference inputs provided.

In some examples, for each of the virtual emissions sensors 571, upon successful auditing of the virtual emissions sensors, the auditor seals the virtual emissions sensor with a virtual seal 521. In some examples, the virtual seal is signed by the auditor with a particular auditor key. In some examples, the particular auditor key is a private key of a public-private key pair that can be verified with the corresponding public key. In some examples, each transaction associated with carbon platform 541 is also signed by the auditor key and is further part of the virtual seal 521. In some examples, the sealing and signature event creating the virtual seal 521 is sent to cross-supply-chain ledger 561 together with the timestamp of the sealing and signature event.

In some examples, the daily aggregated emissions value, the model and the parameters, the location and hash of the archive file with time series data is signed by the auditor key and sent to cross-supply-chain ledger 561 to be stored in cross-supply-chain ledger 561. The precise data which is stored in the local ledger (e.g., ledger 581) versus cross-supply-chain ledger 561, or in both, may vary in different examples. In some examples, data stored in a local ledger can be requested as needed by an authorized entity.

The information stored in the local ledgers (e.g., ledger 581) and cross-supply-chain ledger 561 may be used to determine scope one carbon emissions, scope two carbon emissions, and scope 3 carbon emissions for each of the companies Company 1 through Company N. In some examples, scope one carbon emissions include direct GHG emissions that occur from sources that are controlled or owned by an entity. In some examples, scope two carbon emissions include indirect GHG emissions that are associated with the purchase of electricity, steam, heat, or cooling as a result of the entity's energy use. In some examples, scope three carbons emissions include GHG emissions that result from activities from assets not owned or controlled by the reporting entity, but that the entity indirectly impacts in its supply chain.

In some examples, indirect GHG emissions for a company, for use in determining scope two and scope three emissions, can be determined based on data from the other companies for which there are transactions with the company, as determined by the corresponding transaction IDs. In some examples, system 500 may be used to facilitate the buying and selling of carbon credits among companies Company 1 through Company N. In some examples, for the scope 3 emissions exchange, the supplier provides the emissions value together with the transaction ID, and this provides traceability to the originating calculation for any auditing entities. In some cases, simplified scope 3 emissions may be used where precise scope 3 emissions cannot be determined. For instance, for determining scope 3 emissions that include airplane travel, an average emissions number may be used for airplane travel based on mileage traveled if the precise carbon emissions of the airplane during the particular trip cannot be determined.

In some examples, cross-supply-chain ledger 561 is managed so that authorized entities can access data in cross-supply-chain ledger 561 on demand and in a common format.

In some examples, system 500 may provide third-party auditable certifications and/or audit reports for carbon emissions footprint data provided by the suppliers as scope 3 carbon emissions footprint can be supplied as part of trading messages. In some of these examples, buyers do not need to calculate the emissions and the trading message can be based on the same standards, used readily in the reporting and carbon trading. In some examples, auditor 551 or regulatory authority 552 can trace the emission values back in the supplier's calculation methods, parameters and input parameters and every single footprint calculation may be transparent and auditable by auditor 551, regulatory authority 552, or other suitable authorized third party. In some examples, as part of an audit, signatures may be checked to verify that data has not been changed and to verify that the proper entity made the signature.

In various examples, system 500 may be used for the tracking, reporting, and auditing of carbon emissions. In some examples, the tracking, reporting, and auditing of carbon emissions may be used to meet regulatory requirements, for credible and auditable self-reporting, and/or the like. In some examples, system 500 may also be used to facilitate the buying and selling of carbon credits so that entities can either acquire carbon neutrality or not exceed a carbon emissions cap, and to provide an incentive for entities to sell carbon credits. System 500 may also be used to enables entities to select suppliers with lower carbon emissions, in order to reduce scope two and scope three emissions. In some examples, the tracking of carbon emissions can also be used to detect anomalies, such as leaks. In some examples, the tracking of carbon emissions can be used to assist in optimizing processing, such as reducing carbon emissions and/or improving carbon sequestrations.

FIG. 6 illustrates an example dataflow for a process (690) for externality tracking. In some examples, process 690 is performed by a device, distributed system, or the like, such as, for instance, device 200 of FIG. 2, externality platform device 341 of FIG. 3, externality platform of 441 of FIG. 4, carbon platform 541 of FIG. 5, or the like.

In the illustrated example, step 691 occurs first. At step 691, in some examples, telemetry data that is associated with at least one particular type of quantifiable technical externality is received. As shown, step 692 occurs next in some examples. At step 692, in some examples, auditing of a first virtual sensor by an auditor is enabled. In some examples, the first virtual sensor is configured to output at least a first externality value of a plurality of externality values based on the received telemetry data. In some examples, each externality value of the plurality of externality values is a value that is associated with the at least one particular type of quantifiable technical externality.

As shown, step 693 occurs next in some examples. At step 693, in some examples, signing of the first virtual sensor by the auditor with an auditor key that is associated with the auditor responsive to the auditing of the first virtual sensor being successful is enabled. As shown, step 694 occurs next in some examples. At step 694, in some examples, virtual sensor audit information that is associated with the signing of the first virtual sensor is stored on a first ledger. As shown, step 695 occurs next in some examples. At step 695, in some examples, periodic aggregated externality values are calculated based on the externality values output by the first virtual sensor. As shown, step 696 occurs next in some examples.

At step 696, in some examples, the periodic aggregated externality values are stored on a second ledger. In some examples, the second ledger is a distributed ledger. As shown, step 697 occurs next in some examples. At step 697, in some examples, an audit of the periodic aggregated externality values is enabled. In some examples, the auditing of the periodic aggregated externality values includes verifying the signing of the first virtual sensor by the auditor based, at least in part, on the stored virtual sensor audit information. The process may then advance to a return block, where other processing is resumed.

### CONCLUSION

While the above Detailed Description describes certain examples of the technology, and describes the best mode contemplated, no matter how detailed the above appears in text, the technology can be practiced in many ways. Details may vary in implementation, while still being encompassed by the technology described herein. As noted above, particular terminology used when describing certain features or aspects of the technology should not be taken to imply that the terminology is being redefined herein to be restricted to any specific characteristics, features, or aspects with which that terminology is associated. In general, the terms used in the following claims should not be construed to limit the technology to the specific examples disclosed herein, unless the Detailed Description explicitly defines such terms. Accordingly, the actual scope of the technology encompasses not only the disclosed examples, but also all equivalent ways of practicing or implementing the technology.

We claim:

1. An apparatus, comprising:
at least one memory adapted to store run-time data, and at least one processor that is adapted to execute processor-executable code that, in response to execution, enables the apparatus to perform actions, including:
receiving telemetry data that is associated with at least one particular type of quantifiable technical externality;
enabling auditing of a first virtual sensor by an auditor, wherein the first virtual sensor is configured to output at least a first externality value of a plurality of externality values based on the received telemetry data, and wherein each externality value of the plurality of externality values is a value that is associated with the at least one particular type of quantifiable technical externality;
enabling signing of the first virtual sensor by the auditor with an auditor key that is associated with the auditor responsive to the auditing of the first virtual sensor being successful;
storing, on a first ledger, virtual sensor audit information that is associated with the signing of the first virtual sensor;
calculating periodic aggregated externality values based on the externality values output by the first virtual sensor;
storing, on a second ledger, the periodic aggregated externality values, wherein the second ledger is a distributed ledger; and
enabling an audit of the periodic aggregated externality values, wherein the auditing of the periodic aggregated externality values includes verifying the signing of the first virtual sensor by the auditor based, at least in part, on the stored virtual sensor audit information.

2. The apparatus of claim 1, wherein the first ledger and the second ledger are different ledgers.

3. The apparatus of claim 1, wherein the at least one particular type of quantifiable technical externality includes a quantity of emissions of at least one type of greenhouse gas.

4. The apparatus of claim 1, wherein the at least one particular type of quantifiable technical externality is a carbon equivalent of greenhouse gas emissions.

5. The apparatus of claim 1, the actions further including:
assigning a transaction identifier to the first externality value based on a transaction that is associated with the first externality value; and
storing the transaction identifier along with the periodic aggregated externality values on the second ledger.

6. The apparatus of claim 1, wherein first virtual sensor is further configured to output at least an additional externality value of the plurality of externality values at each time interval of a plurality of time intervals.

7. The apparatus of claim 1, the actions further including: based in part on communication with the second ledger, determining scope one externality values, scope two externality values, and scope three externality values.

8. The apparatus of claim 1, wherein the at least one particular type of quantifiable technical externality includes a quantity of emissions of greenhouse gasses, the actions further including: based in part on communication with the second ledger, determining scope one greenhouse gas emissions values, scope two greenhouse gas emissions values, and scope three greenhouse gas emissions values.

9. The apparatus of claim 1, wherein the first virtual sensor includes a model and a plurality of parameters, and wherein auditing the first virtual sensor includes auditing the model and the plurality of parameters.

10. A method, comprising:
enabling auditing of a first virtual sensor by an auditor, wherein the first virtual sensor is configured to output at least a first externality value of a plurality of externality values based on telemetry data, and wherein each externality value of the plurality of externality values is a value that is associated with the at least one particular type of quantifiable technical externality;
enabling signing of the first virtual sensor by the auditor with an auditor key that is associated with the auditor responsive to the auditing of the first virtual sensor being successful;
storing virtual sensor audit information that is associated with the signing of the first virtual sensor;
via at least one processor, calculating periodic aggregated externality values based on the externality values output by the first virtual sensor; and
communicating the periodic aggregated externality values to a distributed ledger.

11. The method of claim 10, wherein the at least one particular type of quantifiable technical externality includes a quantity of emissions of at least one type of greenhouse gas.

12. The method of claim 10, wherein the at least one particular type of quantifiable technical externality is a carbon equivalent of greenhouse gas emissions.

13. The method of claim 10, further comprising:
assigning a transaction identifier to the first externality value based on a transaction that is associated with the first externality value; and
communicating the transaction identifier along with the periodic aggregated externality values to the second ledger.

14. The method of claim 10, further comprising: based in part on communication with the second ledger, determining scope one externality values, scope two externality values, and scope three externality values.

15. The method of claim 10, wherein the first virtual sensor includes a model and a plurality of parameters, and wherein auditing the first virtual sensor includes auditing the model and the plurality of parameters.

16. A processor-readable storage medium, having stored thereon processor-executable code that, upon execution by at least one processor, enables actions, comprising:
receiving telemetry data that is associated with greenhouse gas emissions;
enabling auditing of a first virtual emissions sensor by an auditor, wherein the first virtual emissions sensor is configured to output at least a first greenhouse gas emissions value of a plurality of greenhouse gas emissions values based on the received telemetry data;
enabling signing of the virtual emissions sensor by the auditor with an auditor key that is associated with the auditor responsive to the auditing of the virtual emissions sensor being successful;
storing virtual emissions sensor audit information that is associated with the signing of the first virtual sensor on a first ledger;
calculating periodic aggregated emissions values based on the greenhouse gas emissions values output by the first virtual sensor;
communicating the periodic aggregated emissions values to a second ledger, wherein the second ledger is a distributed ledger; and
enabling auditing of the periodic aggregated emissions values, wherein the auditing of the periodic aggregated emissions values includes verifying the signing of the virtual emissions sensor by the auditor based, at least in part, on the stored virtual emissions sensor audit information.

17. The processor-readable storage medium of claim 16, the actions further comprising:
assigning a transaction identifier to the first externality value based on a transaction that is associated with the first externality value; and
communicating the transaction identifier along with the periodic aggregated externality values to the second ledger.

18. The processor-readable storage medium of claim 16, wherein first virtual emissions sensor is further configured to output at least an additional greenhouse gas emissions value of the plurality of greenhouse gas emissions values at each time interval of a plurality of time intervals.

19. The processor-readable storage medium of claim 16, the actions further comprising: based in part on communication with the second ledger, determining scope one greenhouse gas emissions values, scope two greenhouse gas emissions values, and scope three greenhouse gas emissions values.

20. The processor-readable storage medium of claim 16, wherein the first virtual sensor includes a model and a plurality of parameters, and wherein auditing the first virtual sensor includes auditing the model and the plurality of parameters.
